(19) United States
(12) Patent Application Publication
(10) Pub. No.: US 2025/0391017 A1
(43) Pub. Date: Dec. 25, 2025

(54) BASE CALLING METHOD AND SYSTEM, GENE SEQUENCER AND STORAGE MEDIUM

(71) Applicant: BGI Shenzhen, Guangdong (CN)

(72) Inventors: Mei Li, Guangdong (CN); Yuxiang Li, Guangdong (CN); Wenwei Zhang, Guangdong (CN); Xun Xu, Guangdong (CN)

(21) Appl. No.: 18/879,715

(22) PCT Filed: Jun. 29, 2022
(86) PCT No.: PCT/CN2022/102503

§ 371 Date: Dec. 27, 2024

(51) Int. Cl. G06T 7/00; G06T 5/40; G06T 5/92; G06T 7/11; G06T 7/90; G06V 10/72; G06V 10/762; G06V 10/764; G06V 20/69; G16B 30/00

(52) U.S. Cl. CPC G06T 7/0012; G06T 5/40; G06T 5/92; G06T 7/11; G06T 7/90; G06V 10/72; G06V 10/763; G06V 20/693; G06V 20/698; G16B 30/00; G06T 2207/10024; G06T 2207/30072; G06V 10/764; G06V 2201/04

(57) ABSTRACT

A base calling method and system, a gene sequencer and a storage medium. The base calling method comprises the following steps: acquiring a first image of a biochip in a red light channel and a second image of the biochip in a green light channel (S1); performing base grouping according to the first image and the second image, and preliminarily identifying the base category of each group (S2); when the number of the base categories of all the groups is at least two, adjusting the brightness value of the first image and the brightness value of the second image according to the base categories of all the groups (S3); respectively performing normalization processing on the first image and the second image (S4); and performing base grouping according to the normalized first image and the normalized second image, and identifying the base category of each group again (S5). The base calling method can accurately identify base categories for data to be sequenced in which some base categories are missing, so that the accuracy of gene sequencing can be improved.

### TECHNICAL FIELD

The present disclosure relates to the field of gene sequencing, in particular to a base calling method and system, a gene sequencer, and a storage medium.

### BACKGROUND

Gene sequencing refers to the analysis of the base sequence of a specific DNA (deoxyribonucleic acid) fragment, specifically the arrangement of adenine (A), thymine (T), cytosine (C), and guanine (G). In general sequencing requirements, the provided data represent balanced bases of four types: A, T, C, and G, with each base roughly accounting for 25% of the total. However, in certain sequencing requirements, the base composition for data to be sequenced may be unbalanced, such as when one or more types of bases are missing.

Existing base calling methods are typically designed for data with balanced base composition and cannot accurately identify base types for data to be sequenced with unbalanced base composition, which leads to gene sequencing failure.

### CONTENT OF THE PRESENT INVENTION

The technical problem addressed by the present disclosure is to overcome the deficiency of existing base calling methods, which cannot accurately identify base types for data to be sequenced with unbalanced base composition. The present disclosure provides a base calling method and system capable of accurately identifying base types for data to be sequenced in which some base types are missing, a gene sequencer, and a storage medium.

A first aspect of the present disclosure provides a base calling method comprising the following steps:
acquiring a first image of a biochip in a red light channel and a second image of the biochip in a green light channel;
performing base grouping according to the first image and the second image, and preliminarily identifying the base type of each group;
adjusting the brightness value of the first image and the brightness value of the second image according to the base types of all the groups;
performing normalization processing on the first image according to a maximum brightness value and a minimum brightness value of the first image, and performing normalization processing on the second image according to a maximum brightness value and a minimum brightness value of the second image;
performing base grouping according to the normalized first image and the normalized second image, and identifying the base type of each group again.

Optionally, the step of performing base grouping according to the first image and the second image, and preliminarily identifying the base type of each group specifically comprises:
calculating a two-dimensional histogram according to the first image and the second image; wherein the axes of the two-dimensional histogram respectively correspond to the brightness value of the first image and the brightness value of the second image;
determining independent regions in the two-dimensional histogram to obtain the base grouping result, wherein each independent region corresponds to a certain group;
determining the radius and angle of each group according to the central position of each group;
preliminarily identifying the base type of each group according to its radius and angle.

Optionally, the step of adjusting the brightness value of the first image and the brightness value of the second image according to the base types of all the groups specifically comprises:
when the number of the base types of all the groups is at least two, if a first base is missing, limiting the minimum brightness value of the first image and the minimum brightness value of the second image, wherein the radius of the group corresponding to the first base is less than a preset value; and/or,
when the number of the base types of all the groups is at least two, if a second base is missing, determining the maximum brightness value of the first image according to the maximum brightness value of the second image, wherein the radius of the group corresponding to the second base is greater than or equal to the preset value, and the angle of the group is less than or equal to a first angle threshold; and/or,
when the number of the base types of all the groups is at least two, if a third base is missing, determining the maximum brightness value of the second image according to the maximum brightness value of the first image, wherein the radius of the group corresponding to the third base is greater than or equal to the preset value, and the angle of the group is greater than or equal to a second angle threshold.

Optionally, the step of performing base grouping according to the normalized first image and the normalized second image, and identifying the base type of each group again specifically comprises:
determining whether a first base is comprised among the base types of all the groups; wherein the radius of the group corresponding to the first base is less than the preset value;
if yes, calculating the radius of each point in the two-dimensional histogram, and determining the point with a radius less than the preset value as belonging to the group corresponding to the first base; identifying the base types of the other groups; if no, directly identifying the base types of the other groups.

Optionally, the preset value is determined according to the following steps:
calculating a radius histogram according to the radius of each point in the two-dimensional histogram;
determining a local maximum and a local minimum in the radius histogram;
determining the two largest local maxima among all the local maxima;
finding the smallest local minimum between the two largest local maxima;
determining the smallest local minimum as the preset value.

Optionally, if the preliminarily identified base types of all the groups include at least two of a second base, a third base, and a fourth base, the step of identifying the base types of the other groups specifically comprises:
calculating an angle of each point belonging to the other groups;
calculating an angle histogram according to the angle of each point in the other groups;
identifying the base types of the other groups according to the angle histogram;
wherein the radius of the group corresponding to the second base is greater than or equal to a preset value, and the angle of the group is less than or equal to a first angle threshold; the radius of the group corresponding to the third base is greater than or equal to the preset value, and the angle of the group is greater than or equal to a second angle threshold; the radius of the group corresponding to the fourth base is greater than or equal to the preset value, and the angle of the group is greater than the first angle threshold and less than the second angle threshold.

Optionally, the step of calculating an angle of each point belonging to the other groups specifically comprises:
if there is a first base comprised among the base types of all the groups, calculating an angle of each point in the other groups according to the central position of the group corresponding to the first base, wherein the radius of the group corresponding to the first base is less than the preset value.

Optionally, the step of identifying the base types of the other groups according to the angle histogram specifically comprises:
determining the position of a valley in the angle histogram;
identifying the base types of the other groups according to the positional relationship between each point and the valley in the angle histogram.

Optionally, following the step of performing base grouping according to the normalized first image and the normalized second image, and identifying the base type of each group again, the base calling method further comprises:
performing clustering analysis on each group according to the re-identified base type of each group to obtain the final base type of each group.

A second aspect of the present disclosure provides a base calling system, comprising:
an image acquisition module, configured to acquire a first image of a biochip in a red light channel and a second image of the biochip in a green light channel;
a preliminary identification module, configured to perform base grouping according to the first image and the second image, and preliminarily identify the base type of each group;
an image processing module, configured to adjust the brightness value of the first image and the brightness value of the second image according to the base types of all the groups;
a normalization module, configured to perform normalization processing on the first image according to a maximum brightness value and a minimum brightness value of the first image, and perform normalization processing on the second image according to a maximum brightness value and a minimum brightness value of the second image;
a secondary identification module, configured to perform base grouping according to the normalized first image and the normalized second image, and identify the base type of each group again.

A third aspect of the present disclosure provides a gene sequencer, comprising a memory, a processor, and a computer program stored in the memory and executable on the processor, wherein the processor, when executing the computer program, implements the base calling method according to the first aspect.

A fourth aspect of the present disclosure provides a computer-readable storage medium, on which a computer program is stored, wherein the computer program, when executed by a processor, implements the base calling method according to the first aspect.

The positive and progressive effects of the present disclosure include: performing preliminary identification of base types according to a first image of a biochip in a red light channel and a second image of the biochip in a green light channel; adjusting the brightness value of the first image and the brightness value of the second image according to the base types of all the groups; performing normalization processing on the first image according to a maximum brightness value and a minimum brightness value of the first image, and performing normalization processing on the second image according to a maximum brightness value and a minimum brightness value of the second image; performing secondary identification of base types according to the normalized first image and the normalized second image.

For data to be sequenced in which some base types are missing, the base calling method provided by the present disclosure can accurately identify base types, so that the accuracy of gene sequencing can be improved. Furthermore, even in cases where some base types are missing, the first and second images can still be normalized, without affecting the subsequent calculation of the Q value, i.e., the quality factor.

### BRIEF DESCRIPTION OF THE DRAWINGS

FIG. 1 is a flowchart illustrating a base calling method provided in Example 1 of the present disclosure.

FIG. 2 is a detailed flowchart of step S2 provided in Example 1 of the present disclosure.

FIG. 3 is a two-dimensional histogram provided in Example 1 of the present disclosure.

FIG. 4 is a two-dimensional histogram following an erosion operation provided in Example 1 of the present disclosure.

FIG. 5 is a schematic diagram of encoding provided in Example 1 of the present disclosure.

FIG. 6 is a detailed flowchart of step S5 provided in Example 1 of the present disclosure.

FIG. 7 is a radius histogram provided in Example 1 of the present disclosure.

FIG. 8 is a diagram illustrating the identification effect of the first base provided in Example 1 of the present disclosure.

FIG. 9 is a diagram illustrating the final identification effect of base types provided in Example 1 of the present disclosure.

FIG. 10 is a structural block diagram of a base calling system provided in Example 1 of the present disclosure.

FIG. 11 is a structural schematic diagram of a gene sequencer provided in Example 2 of the present disclosure.

### DETAILED DESCRIPTION OF THE PREFERRED EMBODIMENT

The present disclosure is further described below through examples, but it is not limited to the scope of these examples.

### Example 1

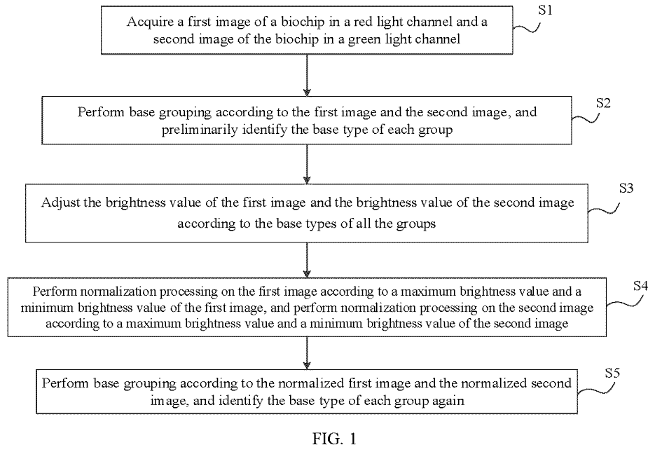

FIG. 1 is a schematic flow diagram of a base calling method provided by this example. The base calling method can be executed by a base calling system, which can be implemented through software and/or hardware. The base calling system may constitute part or all of a gene sequencer.

The base calling method provided by this example is introduced below, using a gene sequencer as the execution entity. As shown in FIG. 1, the base calling method provided by this example may comprise the following steps S1 to S5:

Step S1: acquiring a first image of a biochip in a red light channel and a second image of the biochip in a green light channel.

In a specific embodiment, the gene sequencer is equipped with two laser tubes of red and green wavelengths for emitting red excitation light and green excitation light, respectively, to excite the four bases (A, T, C, and G) in DNA molecules. The biochip forms a first image in the red light channel and a second image in the green light channel. During the process of excitation by the light, these four bases, each tagged with a different fluorescent dye, may emit light or remain non-emissive. In a specific example, the T base only appears in the second image, the C base only appears in the first image, the A base appears in both the first and second images, and the G base does not appear in either the first or second image. In another specific example, the C base only appears in the second image, the T base only appears in the first image, the G base appears in both the first and second images, and the A base does not appear in either the first or second image.

It should be noted that the presence or absence of a base in the image is relative and can be determined specifically based on the grayscale value. For example, if the T base has a grayscale value of 0 in the first image and a grayscale value of 255 in the second image, it can be determined that the T base appears in the second image but not in the first image. Similarly, if the T base has a grayscale value of 2 in the first image and a grayscale value of 254 in the second image, it can also be determined that the T base appears in the second image but not in the first image.

Herein, the biochip mentioned above may also be referred to as a gene chip or a DNA chip.

Step S2: performing base grouping according to the first image and the second image, and preliminarily identifying the base type of each group.

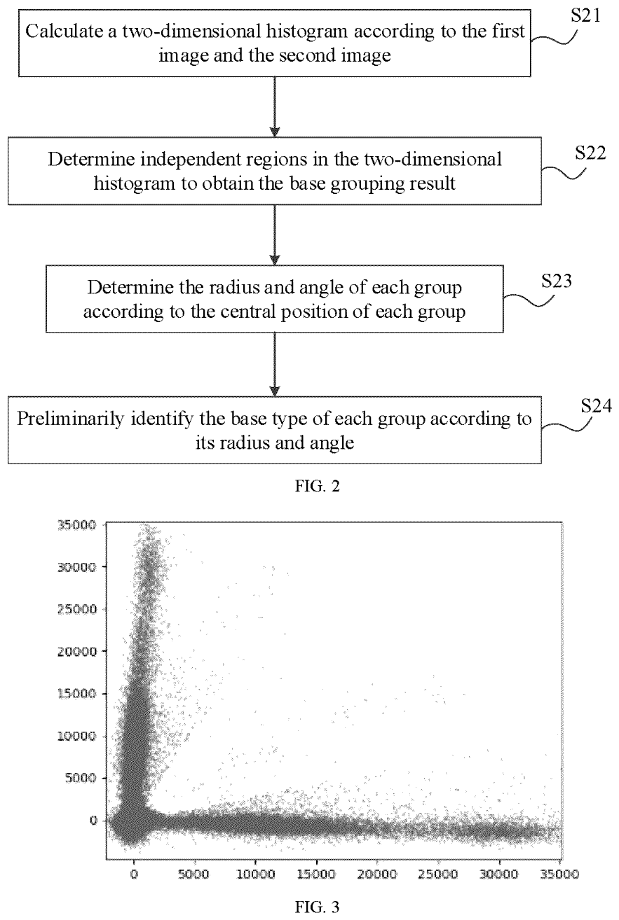

In an optional embodiment, as shown in FIG. 2, the step S2 specifically comprises the following steps S21 to S24:

Step S21: calculating a two-dimensional histogram according to the first image and the second image.

Herein, the axes of the two-dimensional histogram respectively correspond to the brightness value of the first image and the brightness value of the second image. In a specific embodiment, the number of segments on the horizontal and vertical axes of the two-dimensional histogram can be the square root of the number of DNB points. Herein, DNBs (DNA nanoballs) refer to DNA nanoball molecules, with regularly arranged sites (e.g., nanopores) on the biochip. The sites can be arranged in a rectangular grid on the biochip, with each site capable of accommodating or adsorbing a gene cluster (e.g., one DNB or multiple DNA strands with the identical sequence). Using the gene cluster as a template within the site, multiple identical bases are added during each biochemical cycle. The base type at a given site can be determined based on images generated through different light combinations (e.g., the first image and the second image).

In the two-dimensional histogram shown in FIG. 3, the horizontal axis corresponds to the brightness value of the first image, and the vertical axis corresponds to the brightness value of the second image.

In a specific embodiment, to improve the accuracy of preliminary identification of base types, denoising can be performed on the two-dimensional histogram. Specifically, the two-dimensional histogram is sorted in descending order, and the density value at the Pth percentile of the total number of DNBs is identified. All positions in the two-dimensional histogram with values less than the density value are set to 0, thereby removing discrete points from the two-dimensional histogram. Herein, the Pth percentile can be adjusted based on actual requirements, for instance, ranging from P70 to P90. In a specific example, if the total number of DNBs is 100 and the Pth percentile is P70, with a grayscale value of 10 at P70, then all positions in the two-dimensional histogram with values less than 10 are set to 0, resulting in a denoised two-dimensional histogram.

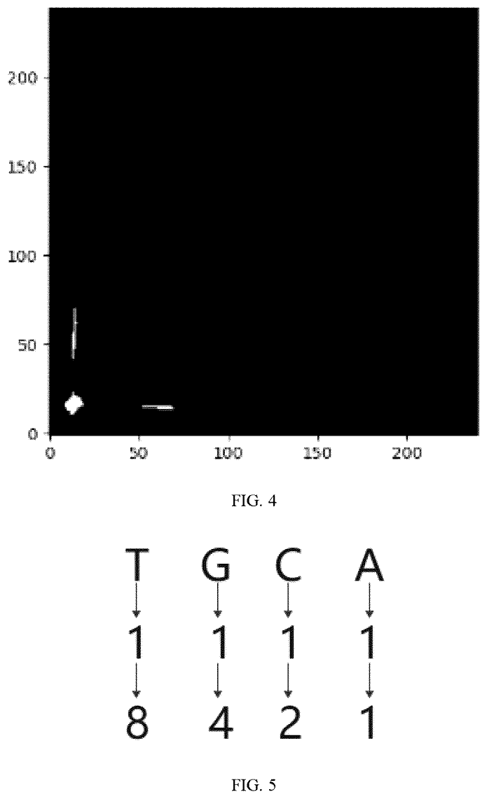

In a specific embodiment, to further improve the accuracy of preliminary identification of base types, an erosion operation can be performed on the denoised two-dimensional histogram. Specifically, all non-zero points in the two-dimensional histogram are set to 1, resulting in a mask. The mask serves as a template for performing point erosion, yielding the result shown in FIG. 4.

Step S22: determining independent regions in the two-dimensional histogram to obtain the base grouping result. Herein, each independent region corresponds to a certain group.

In a specific embodiment, independent regions can be determined based on troughs in the two-dimensional histogram. In some examples, independent regions may also be referred to as a group.

Step S23: determining the radius and angle of each group according to the central position of each group.

In a specific embodiment, the central position of a certain group can be determined based on the average of the horizontal coordinates and the average of the vertical coordinates of all points in the group within the two-dimensional histogram. Herein, to improve the accuracy of calculation, eight-connectivity component labeling can be applied to the group before determining the central position of the group. Further, by converting the coordinates of the two-dimensional histogram to polar coordinates, the radius and angle of the group can be obtained.

Step S24: preliminarily identifying the base type of each group according to its radius and angle.

In a specific embodiment, if the radius of a certain group is less than a preset value, the base type of the group can be identified as the first base. If the radius of a certain group is greater than or equal to the preset value, and the angle is less than or equal to a first angle threshold, then the base type of the group can be identified as the second base. If the radius of a certain group is greater than or equal to the preset value, and the angle is greater than or equal to a second angle threshold, then the base type of the group can be identified as the third base. If the radius of a certain group is greater than or equal to the preset value, and the angle is greater than the first angle threshold but less than the second angle threshold, then the base type of the group can be identified as the fourth base.

In other optional embodiments of step S2, the DBSCAN (Density-Based Spatial Clustering of Applications with Noise) method can also be used for base grouping. Herein, DBSCAN is a density-based clustering method based on regions of high-density connectivity.

In an optional embodiment, following the step S2, the base calling method further comprises: encoding the base types. In a specific example, the first base is G, the second base is C, the third base is T, and the fourth base is A. Binary encoding is used for base types, as shown in FIG. 5: the A base corresponds to bit 0, the C base corresponds to bit 1, the G base corresponds to bit 3, and the T base corresponds to bit 4. Assuming that the preliminarily identified base types include A, C, and T, the binary encoding would be 1011, corresponding to a Flag value of 8+2+1=11. Assuming that the preliminarily identified base types include C and T, the binary encoding would be 1010, corresponding to a Flag value of 8+2=10. In this embodiment, the preliminarily identified base types of all the groups can be subsequently determined by the Flag value.

Step S3: adjusting the brightness value of the first image and the brightness value of the second image according to the base types of all the groups.

In a specific embodiment of step S3, when the number of the base types of all the groups is at least two:

If a first base is missing, the minimum brightness value of the first image and the minimum brightness value of the second image are limited. Herein, the radius of the group corresponding to the first base is less than the preset value. Specifically, both the minimum brightness value of the first image and the minimum brightness value of the second image can be set to a small value, such as 0.

If a third base is missing, the maximum brightness value of the second image is determined according to the maximum brightness value of the first image. Herein, the radius of the group corresponding to the third base is greater than or equal to the preset value, and the angle of the group is greater than or equal to a second angle threshold. For example, the maximum brightness value of the first image can be used as the maximum brightness value of the second image.

If a second base is missing, the maximum brightness value of the first image is determined according to the maximum brightness value of the second image. Herein, the radius of the group corresponding to the second base is greater than or equal to the preset value, and the angle of the group is less than or equal to a first angle threshold. For example, the maximum brightness value of the second image can be used as the maximum brightness value of the first image.

It should be noted that if a fourth base is missing, no adjustments are made to the maximum and minimum brightness values of either the first image or the second image.

Additionally, it should be noted that if the number of the base types of all the groups is only one, the following steps S4 and S5 do not need to be executed.

Step S4: performing normalization processing on the first image according to a maximum brightness value and a minimum brightness value of the first image, and performing normalization processing on the second image according to a maximum brightness value and a minimum brightness value of the second image.

In an optional embodiment of step S4, the first image is normalized according to the following formula:

$$out\_data {\_H}_{P} = ( in\_data {\_H}_{P} - min H ) / ( max H - min H )$$

wherein in_data_H_{P} is the brightness value of the point P in the first image, minH is the minimum brightness value of the first image, maxH is the maximum brightness value of the first image, out_data_H_{P} is the brightness value of the point P in the normalized first image, and the point P represents any point in the first image.

The second image is normalized according to the following formula:

$$out\_data {\_L}_{Q} = ( in\_data {\_L}_{Q} - min L ) / ( max L - min L )$$

wherein in_data_L_{Q} is the brightness value of the point Q in the second image, minL is the minimum brightness value of the second image, maxL is the maximum brightness value of the second image, out_data_L_{Q} is the brightness value of the point Q in the normalized second image, and the point Q represents any point in the second image.

Step S5: performing base grouping according to the normalized first image and the normalized second image, and identifying the base type of each group again.

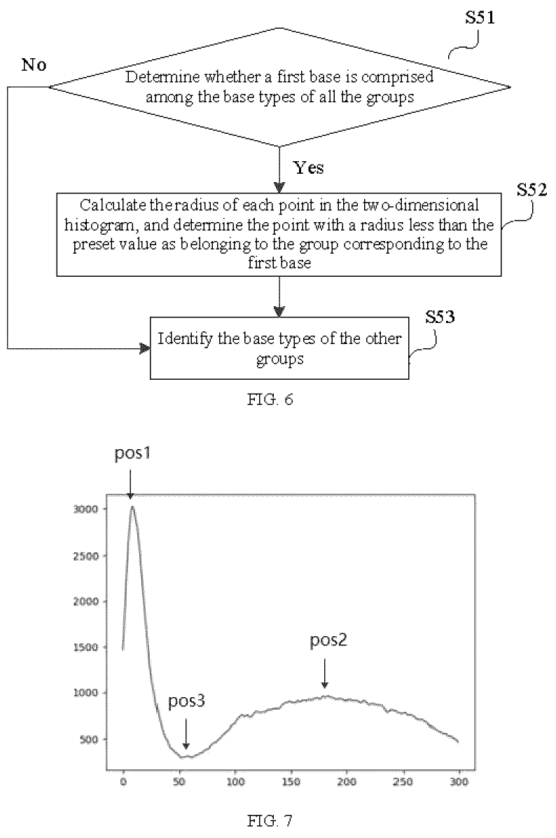

In a specific embodiment, as shown in FIG. 6, step S5 may comprise the following steps S51 to S53:

Step S51: determining whether a first base is comprised among the base types of all the groups; if yes, proceeding to step S52; if no, proceeding to step S53. Herein, the radius of the group corresponding to the first base is less than the preset value.

Step S52: calculating the radius of each point in the two-dimensional histogram, and determining the point with a radius less than the preset value as belonging to the group corresponding to the first base.

In a specific embodiment, the radius R_{M} of the point M in the two-dimensional histogram can be calculated according to the following formula:

$${R}_{M} = \sqrt{{x}_{M}^{2} + {y}_{M}^{2}}$$

wherein x_{M} is the horizontal coordinate of the point M, and y_{M} is the vertical coordinate of the point M.

In an optional embodiment, the preset value is determined according to the following steps S52a to S52e:

Step 52a: calculating a radius histogram according to the radius of each point in the two-dimensional histogram.

In a specific embodiment, the radius histogram for some points in the two-dimensional histogram can be counted. In a specific example, the radius histogram between the P1 percentile and the P99 percentile is counted. Furthermore, multi-point smoothing can be applied to the radius histogram to eliminate spikes in the radius histogram.

Step S52b: determining a local maximum and a local minimum in the radius histogram.

In a specific embodiment, a point can be determined to be a local maximum depending on whether all of its neighboring points are less than the value at the point. If yes, the point is determined to be a local maximum. Similarly, a point can be determined to be a local minimum depending on whether all of its neighboring points are greater than the value at the point. If yes, the point is determined to be a local minimum.

Step S52c: determining the two largest local maxima among all the local maxima.

In a specific embodiment, the interval between the two largest local maxima can be restricted to prevent errors in determining the preset value due to abnormal distribution of the radius histogram. In a specific example, it is required that the interval between the two largest local maxima must be greater than Nth, wherein Nth can be set based on the number of segments N on the horizontal and vertical axes of the radius histogram. For example, if N is 128, Nth is set to 128*20%.

Step S52d: finding the smallest local minimum between the two largest local maxima.

Step S52e: determining the smallest local minimum as the preset value.

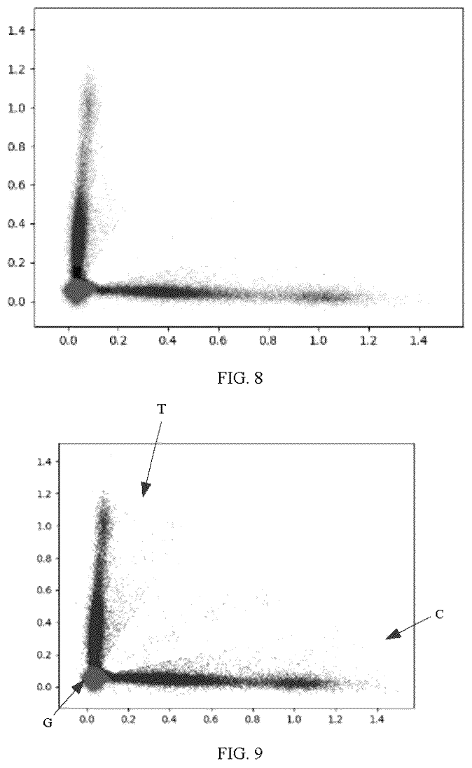

FIG. 7 illustrates a radius histogram. As shown in FIG. 7, the horizontal axis represents the angle, and the vertical axis represents the radius. The two largest local maxima are peak pos1 and peak pos2, respectively. The smallest local minimum between peak pos1 and peak pos2 is identified as valley pos3. In this example, valley pos3 is determined as the preset value described above. In the two-dimensional histogram shown in FIG. 8, the point with a radius less than the preset value is determined as belonging to the group corresponding to the first base. In FIG. 8, the circular independent region corresponds to the group for the first base.

Step S53: identifying the base types of the other groups. Herein, the other groups include the group corresponding to the second base, the group corresponding to the third base, and the group corresponding to the fourth base.

In this embodiment, when the preliminarily identified base types of all the groups have at least two bases and include the first base, the group corresponding to the first base is identified first, followed by the identification of the groups corresponding to other bases.

In an optional embodiment, if the preliminarily identified base types of all the groups include at least two of the second base, the third base, and the fourth base, the step S53 specifically comprises the following steps S53a to S53c:

Step S53a: calculating an angle of each point belonging to the other groups.

In an optional embodiment of step S53a, if there is a first base comprised among the base types of all the groups, an angle of each point in the other groups is calculated according to the central position of the group corresponding to the first base. Assuming that the first base is G, specifically, the angle θⱼ of the point j in the other groups can be calculated according to the following formula:

$${θ}_{j} = arctan ( \frac{{y}_{j} - centerGL}{{x}_{j} - centerGH} ) * 180 / pi$$

wherein xⱼ is the horizontal coordinate of the point j in the two-dimensional histogram, yⱼ is the vertical coordinate of the point j in the two-dimensional histogram, centerGH is the horizontal coordinate of the central position of the group corresponding to the G base, and centerGL is the vertical coordinate of the central position of the group corresponding to the G base. It should be noted that if xⱼ<0, then θⱼ=θⱼ+180.

In another optional embodiment of step S53a, if there is no first base comprised among the base types of all the groups, the angle is calculated directly based on the horizontal and vertical coordinates of each point in the other groups in the two-dimensional histogram.

Step S53b: calculating an angle histogram according to the angle of each point in the other groups.

In a specific embodiment, to improve the accuracy of base identification, multi-point smoothing can be applied to the angle histogram, resulting in a smoothed angle histogram.

Step S53c: identifying the base types of the other groups according to the angle histogram.

Herein, the radius of the group corresponding to the second base is greater than or equal to the preset value, and the angle of the group is less than or equal to a first angle threshold; the radius of the group corresponding to the third base is greater than or equal to the preset value, and the angle of the group is greater than or equal to a second angle threshold; the radius of the group corresponding to the fourth base is greater than or equal to the preset value, and the angle of the group is greater than the first angle threshold and less than the second angle threshold.

In an optional embodiment, the base types of the other groups are identified based on the valley in the angle histogram. The step S53c specifically comprises: determining the position of a valley in the angle histogram, and identifying the base types of the other groups according to the positional relationship between each point and the valley in the angle histogram.

In this embodiment, if the preliminarily identified base types of all the groups include at least two of the second base, the third base, and the fourth base, then there is one valley in the angle histogram; if the preliminarily identified base types of all the groups include all three of the second base, the third base, and the fourth base, then there are two valleys in the angle histogram.

FIG. 9 illustrates the final identification result of base types. In the two-dimensional histogram shown in FIG. 9, there are three groups, corresponding to the G base, C base, and T base, respectively.

In another optional embodiment, the base types of the other groups are identified based on the peak in the angle histogram. The step S53c specifically comprises: determining the position of a peak in the angle histogram, and identifying the base types of the other groups according to the positional relationship between each point and the peak in the angle histogram.

In this embodiment, if the preliminarily identified base types of all the groups include at least two of the second base, the third base, and the fourth base, then there are two peaks in the angle histogram; if the preliminarily identified base types of all the groups include all three of the second base, the third base, and the fourth base, then there are three peaks in the angle histogram.

It should be noted that if the preliminarily identified base types of all the groups include only one of the second base, the third base, and the fourth base, then the step S53 specifically comprises: using the preliminarily identified base types as final, with no need for secondary identification.

To further improve the accuracy of base identification, following the step S5, the base calling method may further comprise: performing clustering analysis on each group according to the re-identified base type of each group to obtain the final base type of each group. Specifically, a GMM (Gaussian Mixture Model) clustering method can be used to perform clustering analysis on each group, thereby obtaining the final base type of each group.

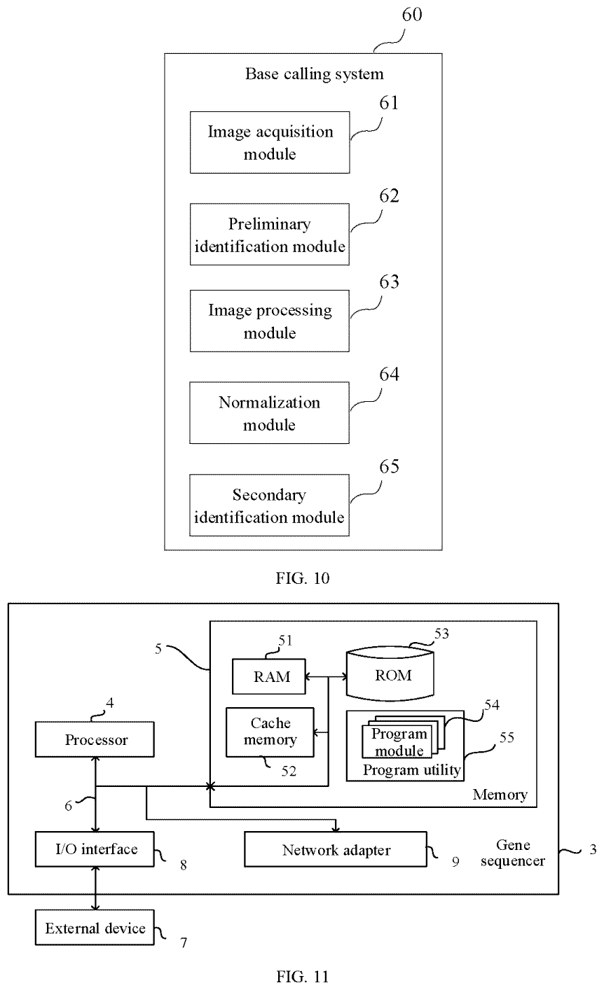

This example further provides a base calling system 60, as shown in FIG. 10, including an image acquisition module 61, a preliminary identification module 62, an image processing module 63, a normalization module 64, and a secondary identification module 65.

The image acquisition module 61 is configured to acquire a first image of a biochip in a red light channel and a second image of the biochip in a green light channel.

The preliminary identification module 62 is configured to perform base grouping according to the first image and the second image, and preliminarily identify the base type of each group.

The image processing module 63 is configured to adjust the brightness value of the first image and the brightness value of the second image according to the base types of all the groups.

The normalization module 64 is configured to perform normalization processing on the first image according to a maximum brightness value and a minimum brightness value of the first image, and perform normalization processing on the second image according to a maximum brightness value and a minimum brightness value of the second image.

The secondary identification module 65 is configured to perform base grouping according to the normalized first image and the normalized second image, and identify the base type of each group again.

In an optional embodiment, the preliminary identification module specifically comprises:
a first calculation unit, configured to calculate a two-dimensional histogram according to the first image and the second image, wherein the axes of the two-dimensional histogram respectively correspond to the brightness value of the first image and the brightness value of the second image;
a first determination unit, configured to determine independent regions in the two-dimensional histogram to obtain the base grouping result, wherein each independent region corresponds to a certain group;
a second determination unit, configured to determine the radius and angle of each group according to the central position of each group;
and a first identification unit, configured to preliminarily identify the base type of each group according to its radius and angle.

In an optional embodiment, the image processing module is specifically configured to, when the number of the base types of all the groups is at least two, if a first base is missing, limit the minimum brightness value of the first image and the minimum brightness value of the second image, wherein the radius of the group corresponding to the first base is less than the preset value.

In an optional embodiment, the image processing module is specifically configured to, when the number of the base types of all the groups is at least two, if a second base is missing, determine the maximum brightness value of the first image according to the maximum brightness value of the second image, wherein the radius of the group corresponding to the second base is greater than or equal to the preset value, and the angle of the group is less than or equal to a first angle threshold.

In an optional embodiment, the image processing module is specifically configured to, when the number of the base types of all the groups is at least two, if a third base is missing, determine the maximum brightness value of the second image according to the maximum brightness value of the first image, wherein the radius of the group corresponding to the third base is greater than or equal to the preset value, and the angle of the group is greater than or equal to a second angle threshold.

In an optional embodiment, the secondary identification module specifically comprises a decision unit, a second calculation unit, and a second identification unit.

The decision unit is configured to decide whether a first base is comprised among the base types of all the groups. If yes, the second calculation unit and the second identification unit are invoked sequentially. If no, the second identification unit is directly invoked. Herein, the radius of the group corresponding to the first base is less than the preset value.

The second calculation unit is configured to calculate the radius of each point in the two-dimensional histogram, and determine the point with a radius less than the preset value as belonging to the group corresponding to the first base.

The second identification unit is configured to identify the base types of the other groups.

In an optional embodiment, the base calling system further comprises a preset value determination module, configured to calculate a radius histogram according to the radius of each point in the two-dimensional histogram; to determine a local maximum and a local minimum in the radius histogram; to determine the two largest local maxima among all the local maxima; to find the smallest local minimum between the two largest local maxima; and to determine the smallest local minimum as the preset value.

In an optional embodiment, if the preliminarily identified base types of all the groups include at least two of the second base, the third base, and the fourth base, then the second identification unit is specifically configured to calculate an angle of each point belonging to the other groups, to calculate an angle histogram according to the angle of each point in the other groups, and to identify the base types of the other groups according to the angle histogram. Herein, the radius of the group corresponding to the second base is greater than or equal to the preset value, and the angle of the group is less than or equal to a first angle threshold; the radius of the group corresponding to the third base is greater than or equal to the preset value, and the angle of the group is greater than or equal to a second angle threshold; the radius of the group corresponding to the fourth base is greater than or equal to the preset value, and the angle of the group is greater than the first angle threshold and less than the second angle threshold.

It should be noted that the base calling system in this example can specifically be a standalone chip, a chip module, or a gene sequencer. Alternatively, it can be a chip or chip module integrated within a gene sequencer.

The various modules/units included in the base calling system described in this example can be software modules/units, hardware modules/units, or a combination of both software and hardware modules/units.

### Example 2

FIG. 11 is a structural schematic diagram of a gene sequencer provided by this example. The gene sequencer comprises at least one processor and a memory communicatively connected to the at least one processor. Herein, the memory stores a computer program executable by the at least one processor, such that when the computer problem is executed, the at least one processor is enabled to execute the base calling method described in Example 1. FIG. 11 shows a gene sequencer 3 as a mere example, which should not impose any limitations on the functions and usage scope of the examples of the present disclosure.

The components of the gene sequencer 3 may include, but are not limited to, at least one processor 4, at least one memory 5, and a bus 6 that connects various system components, including the memory 5 and the processor 4.

The bus 6 comprises a data bus, an address bus, and a control bus.

The memory 5 may comprise a volatile memory, such as a random access memory (RAM) 51 and/or a cache memory 52, and may further comprise a read-only memory (ROM) 53.

The memory 5 may further comprise a program/utility 55 with a set of (at least one) program modules 54 including, but not limited to, an operating system, one or more application programs, other program modules, and program data. Each of these examples or some combination thereof may comprise a component for implementing a network environment.

The processor 4 runs the computer program stored in the memory 5, thereby executing various functional applications and data processing, such as the base calling method described above.

The gene sequencer 3 can also communicate with one or more external devices 7 (e.g., a keyboard or pointing device). Such communication is established through an input/output (I/O) interface 8. Moreover, the gene sequencer 3 can also communicate with one or more networks (e.g., a local area network (LAN), a wide area network (WAN), and/or a public network, such as the Internet) via a network adapter 9. As shown in FIG. 11, the network adapter 9 communicates with other modules of the gene sequencer 3 through the bus 6. It should be understood that although not shown in FIG. 11, other hardware and/or software modules can be used in combination with the gene sequencer 3, including, but not limited to, a microcode, a device driver, a redundant processor, an external disk drive array, a RAID (Redundant Array of Independent Disks) system, a tape drive, and a data backup storage system.

It should be noted that although several units/modules or sub-units/modules of the gene sequencer are mentioned in the detailed description above, such a division is merely exemplary and not mandatory. In practice, according to the embodiments of the present disclosure, the features and functions of two or more units/modules described above may be embodied in a single unit/module. Conversely, the features and functions of a single unit/module described above may be further divided to be embodied by multiple units/modules.

### Example 3

This example provides a computer-readable storage medium storing a computer program, wherein the computer program, when executed by a processor, implements the base calling method described in Example 1.

Herein, the readable storage medium may more specifically include, but is not limited to, a portable disk, a hard disk, a random access memory, a read-only memory, an erasable programmable read-only memory, an optical storage device, a magnetic storage device, or any suitable combination of the above.

In a possible embodiment, the present disclosure may also be implemented in the form of a program product comprising a program code which, when the program product is run on a gene sequencer, causes the gene sequencer to execute the base calling method described in Example 1.

Herein, the program code for executing the present disclosure may be written in any combination of one or more programming languages, and may be executed entirely on the gene sequencer, partially on the gene sequencer, as a standalone software package, partially on the gene sequencer and partially on a remote device, or entirely on a remote device.

Although specific embodiments of the present disclosure have been described above, it should be understood by those skilled in the art that this is merely illustrative, and the scope of protection of the present disclosure is defined by the appended claims. Those skilled in the art may make various changes or modifications to these embodiments without departing from the principles and essence of the present disclosure, but such changes and modifications fall within the scope of protection of the present disclosure.

1. A base calling method, comprising the following steps:
acquiring a first image of a biochip in a red light channel and a second image of the biochip in a green light channel;
performing base grouping according to the first image and the second image, and preliminarily identifying the base type of each group;
adjusting the brightness value of the first image and the brightness value of the second image according to the base types of all the groups;
performing normalization processing on the first image according to a maximum brightness value and a minimum brightness value of the first image, and performing normalization processing on the second image according to a maximum brightness value and a minimum brightness value of the second image;
performing base grouping according to the normalized first image and the normalized second image, and identifying the base type of each group again.

2. The base calling method according to claim 1, wherein the step of performing base grouping according to the first image and the second image, and preliminarily identifying the base type of each group specifically comprises:
calculating a two-dimensional histogram according to the first image and the second image; wherein the axes of the two-dimensional histogram respectively correspond to the brightness value of the first image and the brightness value of the second image;
determining independent regions in the two-dimensional histogram to obtain the base grouping result, wherein each independent region corresponds to a certain group;
determining the radius and angle of each group according to the central position of each group;
preliminarily identifying the base type of each group according to its radius and angle.

3. The base calling method according to claim 2, wherein the step of adjusting the brightness value of the first image and the brightness value of the second image according to the base types of all the groups specifically comprises:
when the number of the base types of all the groups is at least two, if a first base is missing, limiting the minimum brightness value of the first image and the minimum brightness value of the second image, wherein the radius of the group corresponding to the first base is less than a preset value; and/or,
when the number of the base types of all the groups is at least two, if a second base is missing, determining the maximum brightness value of the first image according to the maximum brightness value of the second image, wherein the radius of the group corresponding to the second base is greater than or equal to the preset value, and the angle of the group is less than or equal to a first angle threshold; and/or,
when the number of the base types of all the groups is at least two, if a third base is missing, determining the maximum brightness value of the second image according to the maximum brightness value of the first image, wherein the radius of the group corresponding to the third base is greater than or equal to the preset value, and the angle of the group is greater than or equal to a second angle threshold.

4. The base calling method according to claim 2, wherein the step of performing base grouping according to the normalized first image and the normalized second image, and identifying the base type of each group again specifically comprises:
determining whether a first base is comprised among the base types of all the groups; wherein the radius of the group corresponding to the first base is less than a preset value;
if yes, calculating the radius of each point in the two-dimensional histogram, and determining the point with a radius less than the preset value as belonging to the group corresponding to the first base; identifying the base types of the other groups;
if no, directly identifying the base types of the other groups.

5. The base calling method according to claim 3, wherein the preset value is determined according to the following steps:
calculating a radius histogram according to the radius of each point in the two-dimensional histogram;
determining a local maximum and a local minimum in the radius histogram;
determining the two largest local maxima among all the local maxima;
finding the smallest local minimum between the two largest local maxima;
determining the smallest local minimum as the preset value.

6. The base calling method according to claim 4, wherein if the preliminarily identified base types of all the groups comprise at least two of a second base, a third base, and a fourth base, the step of identifying the base types of the other groups specifically comprises:
calculating an angle of each point belonging to the other groups;
calculating an angle histogram according to the angle of each point in the other groups;
identifying the base types of the other groups according to the angle histogram;
wherein the radius of the group corresponding to the second base is greater than or equal to a preset value, and the angle of the group is less than or equal to a first angle threshold; the radius of the group corresponding to the third base is greater than or equal to the preset value, and the angle of the group is greater than or equal to a second angle threshold; the radius of the group corresponding to the fourth base is greater than or equal to the preset value, and the angle of the group is greater than the first angle threshold and less than the second angle threshold.

7. The base calling method according to claim 6, wherein the step of calculating an angle of each point belonging to the other groups specifically comprises:
if there is a first base comprised among the base types of all the groups, calculating an angle of each point in the other groups according to the central position of the group corresponding to the first base;
wherein the radius of the group corresponding to the first base is less than the preset value.

8. The base calling method according to claim 6, wherein the step of identifying the base types of the other groups according to the angle histogram specifically comprises:
determining the position of a valley in the angle histogram;
identifying the base types of the other groups according to the positional relationship between each point and the valley in the angle histogram.

9. The base calling method according to claim 1, wherein following the step of performing base grouping according to the normalized first image and the normalized second image, and identifying the base type of each group again, the base calling method further comprises:
performing clustering analysis on each group according to the re-identified base type of each group to obtain the final base type of each group.

10. (canceled)

11. A gene sequencer, comprising a memory, a processor, and a computer program stored in the memory and executable on the processor, wherein the processor, when executing the computer program, implements a base calling method;
the base calling method, comprising the following steps:
acquiring a first image of a biochip in a red light channel and a second image of the biochip in a green light channel;
performing base grouping according to the first image and the second image, and preliminarily identifying the base type of each group;
adjusting the brightness value of the first image and the brightness value of the second image according to the base types of all the groups;
performing normalization processing on the first image according to a maximum brightness value and a minimum brightness value of the first image, and performing normalization processing on the second image according to a maximum brightness value and a minimum brightness value of the second image;
performing base grouping according to the normalized first image and the normalized second image, and identifying the base type of each group again.

12. A computer-readable storage medium, on which a computer program is stored, wherein the computer program, when executed by a processor, implements a base calling method;
the base calling method, comprising the following steps:
acquiring a first image of a biochip in a red light channel and a second image of the biochip in a green light channel;
performing base grouping according to the first image and the second image, and preliminarily identifying the base type of each group;
adjusting the brightness value of the first image and the brightness value of the second image according to the base types of all the groups;
performing normalization processing on the first image according to a maximum brightness value and a minimum brightness value of the first image, and performing normalization processing on the second image according to a maximum brightness value and a minimum brightness value of the second image;
performing base grouping according to the normalized first image and the normalized second image, and identifying the base type of each group again.

13. The base calling method according to claim 4, wherein the preset value is determined according to the following steps:
calculating a radius histogram according to the radius of each point in the two-dimensional histogram;
determining a local maximum and a local minimum in the radius histogram;
determining the two largest local maxima among all the local maxima;
finding the smallest local minimum between the two largest local maxima;
determining the smallest local minimum as the preset value.

14. The gene sequencer according to claim 10, wherein the step of performing base grouping according to the first image and the second image, and preliminarily identifying the base type of each group specifically comprises:
calculating a two-dimensional histogram according to the first image and the second image; wherein the axes of the two-dimensional histogram respectively correspond to the brightness value of the first image and the brightness value of the second image;
determining independent regions in the two-dimensional histogram to obtain the base grouping result, wherein each independent region corresponds to a certain group;
determining the radius and angle of each group according to the central position of each group;
preliminarily identifying the base type of each group according to its radius and angle.

15. The gene sequencer according to claim 13, wherein the step of adjusting the brightness value of the first image and the brightness value of the second image according to the base types of all the groups specifically comprises:
when the number of the base types of all the groups is at least two, if a first base is missing, limiting the minimum brightness value of the first image and the minimum brightness value of the second image, wherein the radius of the group corresponding to the first base is less than a preset value; and/or,
when the number of the base types of all the groups is at least two, if a second base is missing, determining the maximum brightness value of the first image according to the maximum brightness value of the second image, wherein the radius of the group corresponding to the second base is greater than or equal to the preset value, and the angle of the group is less than or equal to a first angle threshold; and/or,
when the number of the base types of all the groups is at least two, if a third base is missing, determining the maximum brightness value of the second image according to the maximum brightness value of the first image, wherein the radius of the group corresponding to the third base is greater than or equal to the preset value, and the angle of the group is greater than or equal to a second angle threshold.

16. The gene sequencer according to claim 13, wherein the step of performing base grouping according to the normalized first image and the normalized second image, and identifying the base type of each group again specifically comprises:
determining whether a first base is comprised among the base types of all the groups; wherein the radius of the group corresponding to the first base is less than a preset value;
if yes, calculating the radius of each point in the two-dimensional histogram, and determining the point with a radius less than the preset value as belonging to the group corresponding to the first base; identifying the base types of the other groups;
if no, directly identifying the base types of the other groups.

17. The gene sequencer according to claim 14, wherein the preset value is determined according to the following steps:
calculating a radius histogram according to the radius of each point in the two-dimensional histogram;
determining a local maximum and a local minimum in the radius histogram;
determining the two largest local maxima among all the local maxima;
finding the smallest local minimum between the two largest local maxima;
determining the smallest local minimum as the preset value.

18. The computer-readable storage medium according to claim 11, wherein the step of performing base grouping according to the first image and the second image, and preliminarily identifying the base type of each group specifically comprises:
calculating a two-dimensional histogram according to the first image and the second image; wherein the axes of the two-dimensional histogram respectively correspond to the brightness value of the first image and the brightness value of the second image;
determining independent regions in the two-dimensional histogram to obtain the base grouping result, wherein each independent region corresponds to a certain group;
determining the radius and angle of each group according to the central position of each group;
preliminarily identifying the base type of each group according to its radius and angle.

19. The computer-readable storage medium according to claim 17, wherein the step of adjusting the brightness value of the first image and the brightness value of the second image according to the base types of all the groups specifically comprises:
when the number of the base types of all the groups is at least two, if a first base is missing, limiting the minimum brightness value of the first image and the minimum brightness value of the second image, wherein the radius of the group corresponding to the first base is less than a preset value; and/or,
when the number of the base types of all the groups is at least two, if a second base is missing, determining the maximum brightness value of the first image according to the maximum brightness value of the second image, wherein the radius of the group corresponding to the second base is greater than or equal to the preset value, and the angle of the group is less than or equal to a first angle threshold; and/or,
when the number of the base types of all the groups is at least two, if a third base is missing, determining the maximum brightness value of the second image according to the maximum brightness value of the first image, wherein the radius of the group corresponding to the third base is greater than or equal to the preset value, and the angle of the group is greater than or equal to a second angle threshold.

20. The computer-readable storage medium according to claim 17, wherein the step of performing base grouping according to the normalized first image and the normalized second image, and identifying the base type of each group again specifically comprises:
determining whether a first base is comprised among the base types of all the groups; wherein the radius of the group corresponding to the first base is less than a preset value;
if yes, calculating the radius of each point in the two-dimensional histogram, and determining the point with a radius less than the preset value as belonging to the group corresponding to the first base; identifying the base types of the other groups;
if no, directly identifying the base types of the other groups.

21. The computer-readable storage medium according to claim 18, wherein the preset value is determined according to the following steps:
calculating a radius histogram according to the radius of each point in the two-dimensional histogram;
determining a local maximum and a local minimum in the radius histogram;
determining the two largest local maxima among all the local maxima;
finding the smallest local minimum between the two largest local maxima;
determining the smallest local minimum as the preset value.
